(19) United States
(12) Patent Application Publication
(10) Pub. No.: US 2015/0223880 A1
(43) Pub. Date: Aug. 13, 2015

(54) METHOD FOR THE TREATMENT OF BENIGN PROSTATIC HYPERPLASIA

(71) Applicant: Samuel FARAN, Boernsen (DE)

(72) Inventor: Samuel FARAN, Boernsen (DE)

(73) Assignee: OLYMPUS WINTER & IBE GMBH, Hamburg (DE)

(21) Appl. No.: 14/175,655

(22) Filed: Feb. 7, 2014

(51) Int. Cl. A61B 18/18; A61B 18/14

(52) U.S. Cl. CPC A61B 18/18; A61B 18/1492

(57) ABSTRACT

A method for the treatment of benign prostatic hyperplasia includes ablating at least a portion of one or more veins in the testicular drainage system in a human. For example, at least a portion of one or more of the internal spermatic veins in a human male may be ablated using a radiofrequency device.

### BACKGROUND

Benign prostatic hyperplasia (BPH), also called benign enlargement of the prostate (BEP) and adenofibromyomatous hyperplasia, is an increase in size of the prostate. In BPH, the increase in size of the prostate involves hyperplasia (an increase in the number of cells) rather than hypertrophy (a growth in the size of individual cells).

BPH involves hyperplasia of prostatic stromal and epithelial cells, resulting in the formation of large, fairly discrete nodules in the periurethral region of the prostate. When sufficiently large, the nodules compress the urethral canal to cause partial, or sometimes virtually complete, obstruction of the urethra, which interferes with the normal flow of urine. It leads to symptoms of urinary hesitancy, frequent urination, increased risk of urinary tract infections, urinary retention, and may contribute to or cause insomnia. Although prostate specific antigen levels may be elevated in these patients because of increased organ volume and inflammation due to urinary tract infections, BPH generally does not lead to cancer or increase the risk of cancer.

Adenomatous prostatic growth is believed to begin at approximately age 30 years. An estimated 50% of men have histologic evidence of BPH by age 50 years and 75% by age 80 years; in 40-50% of these men, BPH becomes clinically significant.

Previous methods for the treatment of BPH include drug therapy, such as taking alpha blockers and/or 5α-reductase inhibitors, venography and/or sclerotherapy of the internal spermatic vein network, or surgical methods that remove a portion of the prostate, or remove the prostate completely. However, previous methods have potential side effects, are invasive, and have a potential for complications.

Therefore, there is need to develop improved methods for the treatment of BPH that are minimally invasive, reduce the potential side effects, and reduce the chance of complications from surgery.

### SUMMARY

Described herein is a method, wherein the method comprises identifying a male in need of ablation of at least a portion of one or more veins of a testicular drainage system; and ablating at least a portion of one or more veins of the testicular drainage system of a human.

Also described is a method for the treatment of benign prostatic hyperplasia, the method comprising radiofrequency ablating a least a portion of at least one vein of the testicular drainage system in a male suffering from benign prostatic hyperplasia.

A method for the treatment of hyperplasia of the prostate is described, wherein the method comprises ablating at least a portion of at least one vein of the testicular drainage system in a male suffering from hyperplasia of the prostate, wherein the ablation is performed using a radiofrequency ablation device.

### BRIEF DESCRIPTION OF THE DRAWINGS

FIG. 1 schematically illustrates a typical testicular and prostate venous drainage system of a human male.

FIG. 2 schematically illustrates typical testicular and prostate venous drainage paths in a normal left side of a human male.

FIG. 3 schematically illustrates typical testicular and prostate venous drainage paths in a left side of a human male when the one-way valves in the internal spermatic vein do not function.

FIG. 4A schematically illustrates a guide-wire designed to move through venous valves and junctions, having an expandable and contractible element in a collapsed state.

FIG. 4B schematically illustrates a guide-wire designed to move through venous valves and junctions, having an expandable element in an expanded state.

FIG. 4C schematically illustrates a side view of a distal end of a guide-wire (similar to that of FIGS. 4A and 4B), with expandable and contractible elements, connected to elastic members in a collapsed state.

FIG. 4D schematically illustrates a side view of a distal end of a guide-wire (similar to that of FIGS. 4A and 4B), with expandable and contractible elements, connected to elastic members in an expanded state.

FIG. 5 schematically illustrates a radiofrequency electrode inserted into an internal spermatic vein.

### DETAILED DESCRIPTION

“Testicular drainage system” as used herein refers, for example, to one or more veins that carry blood away from a testicle to the systemic circulatory system. For example, the term “testicular drainage system” may collectively refer to the veins of the pampiniform plexus, the scrotal vein, the deferential vein, cremasteric vein, and the internal spermatic vein. The term “testicular drainage system” does not include, for example, the common iliac, the inferior vena cava, the renal vein, and the veins of the prostatic venous drainage system, for example, the vesicular vein, internal iliac, the veins of the vesicular plexus, or the prostatic venous plexus.

“Systemic circulatory system” as used herein refers to blood vessels of the general cardiovascular system which carries oxygenated blood away from the heart to the body, and returns deoxygenated blood back to the heart.

“Peripheral blood” as used herein refers to blood in the systemic circulatory system.

As used herein, the modifier “about” used in connection with a quantity is inclusive of the stated value and has the meaning dictated by the context (for example, it includes at least the degree of error associated with the measurement of the particular quantity). When used in the context of a range, the modifier “about” should also be considered as disclosing the range defined by the absolute values of the two endpoints. For example, the range “from about 2 to about 4” also discloses the range “from 2 to 4.”

Described herein is a method, wherein the method comprises identifying a male in need of ablation of one or more veins of the testicular drainage system, and ablating at least a portion of one or more veins of the testicular drainage system of a human. The method may be used to treat, for example, hyperplasia of the prostate, such as, for example, benign prostatic hyperplasia.

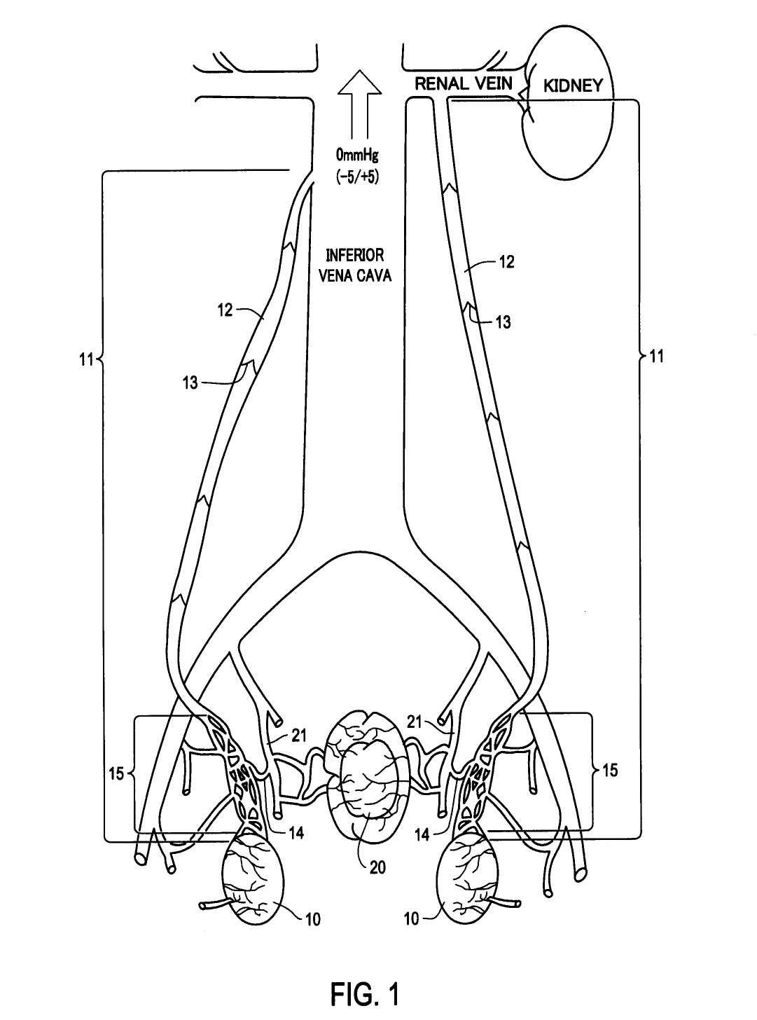

As depicted in FIG. 1, in a healthy male, the testes 10 are the production site of sperm and free testosterone (FT). The testes 10 drain their waste products and free testosterone (FT) through the testicular venous drainage system 11, which includes, among other things, the internal spermatic veins (ISV) 12, to the systemic circulation, diluting the testosterone concentration by about 70 to 100 fold. Sex hormone-binding globulin (SHBG) circulating in the blood binds to FT, thus reducing the bioavailability of the FT. Only a small fraction, about 2%, of the testosterone produced by the testes remains free in the circulating blood. In a healthy individual, the FT reaches the prostate 20 via the circulating blood through the prostate artery. Once in the prostate, FT diffuses into the cytoplasm of the prostatic cells, wherein approximately 90% of the FT is irreversibly converted to dihydrotestosterone (DHT), a more potent androgenic hormone that is involved in the proliferation of prostate cells. Thus, FT is a known promoter of prostate cell proliferation.

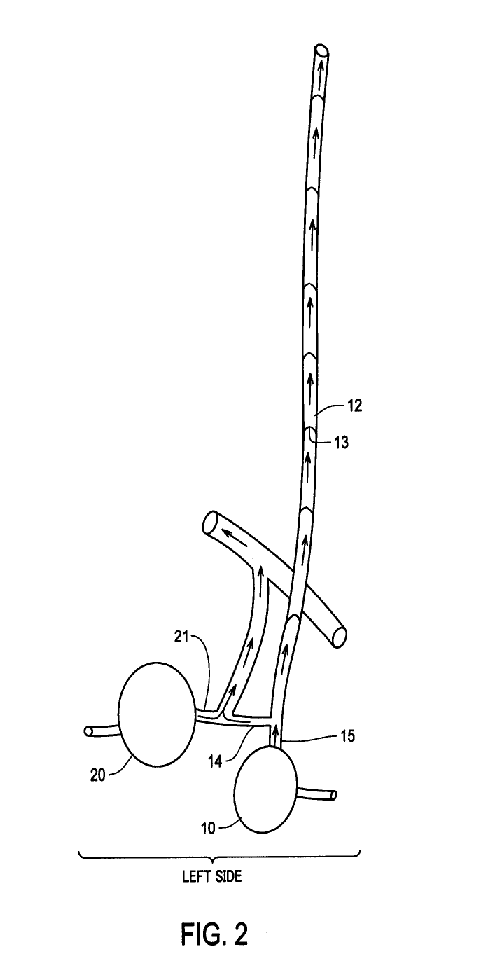

As discussed above, the testes 10 drain their waste products and FT through the testicular venous drainage system 11. The testicular drainage system includes, among other things, the pampiniform plexus 15, which drains into, among other things, the ISVs 12. The left ISV drains into the left renal vein, and the right ISV drains into the inferior vena cava, thus entering into the systemic circulation. The testicular venous drainage system, and in particular, the ISVs 12, facilitate venous blood flow upwards against gravity. Because there is no “active pump” in the testicular venous drainage system, the ISVs comprise a series of one-way valves 13 to (1) prevent the back flow of blood (which contains the waste products and FT), and (2) reduce the overall pressure needed to drain the waste products and the FT from the testes. In a healthy individual, the one-way valves divide the ISVs into about 6-8 individual compartments. Dividing the ISVs into smaller individual compartments reduces the overall hydrostatic pressure in the ISVs and limits the hydrostatic pressure in each compartment to about 4-6 mmHg. FIG. 2 depicts a healthy individual's testicular and prostate venous drainage paths in a normal left side of a human male with arrows to illustrate the venous blood flow as described above.

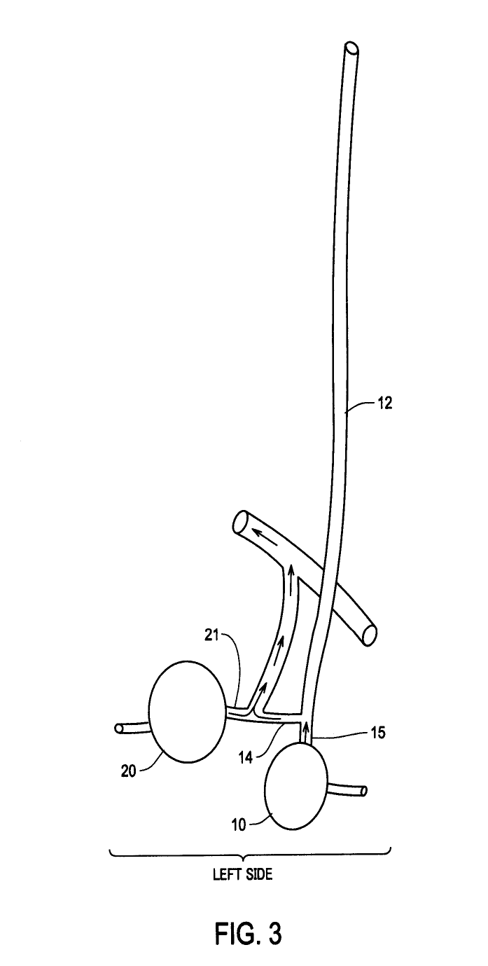

Benign prostatic hyperplasia (BPH) has been associated with the destruction of, or otherwise non-functioning, one-way valves in the testicular venous drainage system, and in the ISVs in particular. As shown in FIG. 3, the destruction of the one-way valves 13 in the ISV 12 causes an increase in the hydrostatic pressure in the ISV that propagates throughout the testicular venous drainage system. For example, when the one-way valves 13 are destroyed, the ISVs become one long continuous column, thus increasing the hydrostatic pressure in the testicular venous drainage system about five to eight times that of the normal hydrostatic pressure in the testicular venous drainage system. Because of this increased column length and increased pressure needed to move the waste products and FT from the testes 10 against gravity, the flow of the waste products and FT upwards against gravity is impaired. Drainage from the testes 10 is then diverted to other channels because of the increased pressure.

One of these other channels is the deferential vein 14. The deferential vein 14 is hydraulically connected to the prostatic venous drainage system, which drains waste products from the prostate 20 via the vesicular vein 21, among other veins. The vesicular vein 21 is connected to the deferential vein 14, thus physically connecting the prostatic venous drainage system with the testicular venous drainage system. Because of this connection, any change in the pressure of one system will cause change of pressure and change in the direction of the flow in the other system. Thus, when the one-way valves 13 are destroyed, and the pressure in the testicular venous drainage system increases, the increase in pressure causes a back-flow of the waste products and FT from the testes into the prostate 20 via the deferential vein 14 and the vesicular vein 21. Thus, not only is FT entering the prostate 20 through the normal physiological method described previously, but also pathologically through the prostate venous drainage system, as depicted in FIG. 3. In a person whose one-way valves 13 have been destroyed, the concentration of total testosterone reaching the testicular drainage system is about 100-fold higher than a healthy individual, and the FT levels in the testicular drainage system reaches about 120 to about 150 times the normal value in the peripheral blood.

It has been found that when one or more of the one-way valves 13 have been destroyed, back-flow into the prostate may be reduced and/or prevented by closing off at least a portion of one or more of the veins of the testicular drainage system. The closing off of at least a portion of one or more of veins in the testicular drainage system may be performed, for example, by ablation of a least a portion of at least one of the veins of the testicular drainage system. Ablating one or more of the veins of the testicular drainage system results in closure of the ablated vein. Radiofrequency ablation of a varicose vein involves using radiofrequency energy to heat the wall of the vein so that it collapses. This causes the vein to close and seal up. Blood is redirected through nearby healthy veins as a result

For example, in order to reduce and/or prevent the back-flow into the prostate, at least a portion of one or more of the ISVs, deferential veins, and/or any other vein that carries FT from the testicle to the prostate, for example, a bypass vein that may have developed as a result of the increased hydrostatic pressure, may be ablated. The ablation may be applied in the opening of the vein or at a spot or region along the vein so that blood cannot flow in or through the vessel. Waste products and FT would then drain out of the testes through other veins in the testicular drainage system. Eliminating the back-flow of FT into the prostate is thus one method to treat and/or prevent BPH.

The ablation of at least a portion of at least one vein of the testicular drainage system may be applied to only the side in which the hydrostatic pressure is increased. For example, if the left testicular drainage system has a normal hydrostatic pressure, and the right testicular drainage system has a higher hydrostatic pressure, such that there is back-flow of FT from the right testicle into the prostate, the ablation may be applied to at least a portion of one or more veins of the right testicular drainage system. Alternatively, if the right testicular drainage system has a normal hydrostatic pressure, and the left testicular drainage system has a higher hydrostatic pressure, such that there is back-flow of FT from the left testicle into the prostate, the ablation may be applied to at least a portion of one or more veins of the left testicular drainage system. The ablation may also be applied to both sides of the body, even if only one side of the testicular drainage system has increased hydrostatic pressure and the other testicular drainage system has a normal hydrostatic pressure.

The ablation may be performed in a person exhibiting symptoms of BPH, or may be applied as a preventative treatment, for example, in a person at a high risk of developing BPH, for example, in a male with a family history of developing BPH, or in males aged 40 and above, for example, aged 50 and above, or aged 60 and above. The ablation may also be performed in a person with elevated hydrostatic pressure in one or more of the testicular drainage systems, or in a person whose testosterone concentration in the testicular drainage system is elevated compared to a normal person, even though that person is not exhibiting symptoms of BPH.

The hydrostatic pressure in one or more of the ISVs may be measured, for example, by introducing into the ISVs a needle or a catheter that is connected to a pressure transducer, which is connected to an amplifier-recorder system which allows the recording of physical changes in the ISVs pressure.

As an alternative to, or in conjunction with, measuring the hydrostatic pressure in one or more of the ISVs, the testosterone concentration may be measured near a testicle, the prostate, or between them for example, in the vesicular vein 21. The testosterone may also be measured at the lower part of the ISV 12, at or above pampiniform plexus 15, the deferential vein 14, and/or vesicular plexus of the prostate 20. The testosterone level may be measured using a syringe, a catheter, and/or by non-invasive methods, for example, radiation, for example using laser or infrared radiation. The FT concentration may be measured, the total testosterone concentration may be measured, or both the FT and total testosterone concentration may be measured. The measurement may be repeated, or a plurality of measurements may be taken at a plurality of anatomical locations. Testosterone may also be tested in the peripheral blood circulation so that a difference between the testosterone in the peripheral blood circulation and in the testicular drainage system and/or the prostatic drainage system may be determined, and may be assessed for diagnosis of BPH. After the measurements, a range of normal and pathological concentrations may be compared to the measured values for diagnosis of BPH, and/or to determine if the one-way valves in the testicular drainage system have been destroyed.

The ablation of at least a portion of one or more of veins of the testicular drainage system may be performed by any suitable method. For example, the ablation may be performed by applying energy to the inside of one or more veins of the testicular drainage system. While energy can be applied from outside of the veins with similar result, it is difficult to place an energy applicator outside of and in close proximity to a vein. The energy may be in the form of, for example, laser energy, microwave energy, a direct current, alternating current, or any other form of energy that generates a sufficient amount of heat to ablate at least a portion of one or more veins of the testicular drainage system. The alternating current may be a high frequency alternating current in the range of, for example, about 30 kHz to about 30 GHz, about 100 kHz to about 10 GHz, or from about 200 kHz to about 4 MHz. For example, the alternating current may be in the range of about 300 kHz to about 600 kHz. The alternating current may be applied using a radiofrequency (RF) device. In other words, the ablation may be in the form of radiofrequency ablation.

The radiofrequency ablation device may be any device that is capable of applying a sufficient amount of alternating current to ablate at least a portion of one or more veins of the testicular drainage system. Radiofrequency ablation devices are known in the art, and are described, for example, in U.S. Pat. No. 6,635,054, U.S. Pat. No. 7,837,676, U.S. Pat. No. 7,241,292, and U.S. Pat. No. 6,152,899, and U.S. Pat. No. 8,452,422, the disclosures of which are incorporated herein by reference.

For example, conventional ablation techniques use an RF probe that may be comprised of a flexible catheter of electrically insulating material, which carries at its distal tip two circumferentially arranged electrodes, which are spaced apart in longitudinal direction. The electrodes are connected to an RF generator by electrical wires, which may either be placed along the inside or the outside of the catheter. The catheter may comprise at least one internal lumen, which can be flushed with cooling liquid during the application of RF energy, to prevent blood sticking to the electrodes. In operation, RF current flows from one electrode through the tissue of the vein's wall to the other electrode, thereby heating the tissue and causing ablation of the vein. RF ablation occurs when a high frequency alternating current flows from one electrode to another, completing a current path, causing ionic agitation. Ionic agitation occurs around an active electrode as a result of frictional heating in the tissue surrounding the electrode, leading to cell death and necrosis. After ablating the target tissue, the flexible catheter carrying the electrodes is removed from the target area.

RF ablation probes may be configured in either monopolar or bipolar mode. In monopolar mode, one electrode, for example a negative electrode, is located within or on a cannula. In order to complete the circuit for RF energy, a separate electrode, for example, an electrode pad or the like, may be placed on the skin of the patient. Other bipolar-based devices use multiple electrodes or electrode arrays on a single device. For example, two electrically independent opposing arrays may be contained within an insulated cannula. RF energy passes between the two arrays and heats the tissue surrounding and in between the arrays. A bipolar configuration is generally preferred with the method described herein.

As discussed above, the RF probe may be guided through the veins and/or arteries of the circulatory system to reach the veins of the testicular drainage system. In order to access the ISV 12 and below, for example, the pampiniform plexus 15 or the deferential vein 14, a RF probe should pass the one-way valves 13 which normally resist flow against the normal flow direction. The right testicular drainage system may be accessed by guiding the RF probe through the right internal spermatic vein which connects to the inferior vena cava at an angle, such that a catheter being advanced from below, for example, from the femoral vein, needs to take a sharp turn to access the right ISV. The RF probe may enter the left ISV via the left renal vein.

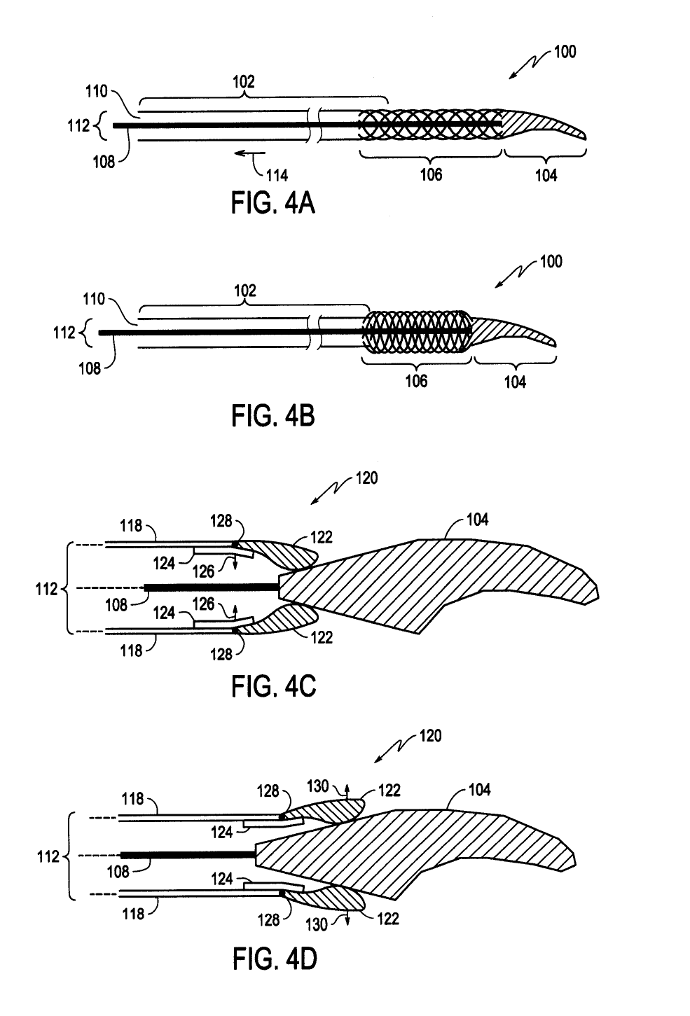

In order to guide an RF probe to the left or right testicular drainage systems, a guide-wire may be used to open the one-way valves and to guide the RF probe sufficiently close to the portion(s) of the vein(s) to be ablated. “Sufficiently close” refers, for example, to a distance close enough to the portion of the vein such that when current is applied by the RF probe, the desired portion of the vein to be ablated is ablated. FIG. 4A schematically illustrates a guide-wire 100 designed to move through venous valves and junctions. The guide-wire may have an expandable and contractible element 106. FIG. 4A schematically illustrates the expandable and contractible element 106 in a collapsed state, and FIG. 4B schematically illustrates the guide-wire 100 having an expandable element 106 in an expanded state.

The guide-wire 100 may comprise an expandable and contractible element 106 near the distal end of guide-wire 100 and an extension at the distal end of element 106, forming a flexible tip 104. The guide-wire 100 further comprises an elongated duct 102 having a lumen 110. The guide-wire 100 may comprise a control-wire 108 that passes through lumen 110. The control wire 108 may pass through the expandable element 106 for expanding and contracting element 106.

Operation of the guide-wire comprises manipulating tip 104 to maneuver the guide-wire 100 to reach a vein near a one-way valve 13, for example, about 2-3 cm upstream of the one-way valve, pulling the control wire 108 from the proximal end of guide-wire 100 in direction 114, thus the pushing distal end (tip 104) against element 106 and compelling element 106 to expand against the walls of the vein. As element 106 expands, it stretches and widens the vein, thereby opening the orifice of the nearby one-way valve 13. Then, the control wire 108 is pushed, which collapses element 106, while guide-wire 100 passes through the valve orifice before it constricts back.

Optionally, an intravascular catheter (not shown) may be maneuvered to the proximity of a one-way valve, and the operator injects a contrast agent via the intravascular catheter to visualize the valve and the position of the catheter. Once the operator is satisfied that the intended position is reached, the guide-wire is inserted in the catheter and manipulated, aided by the tip 104 to reach near a one-way valve 13. The expandable and contractible element 106 is expanded as described above, thus opening the vessel walls and the one-way valve. The control wire is operated as describe above. Once the guide-wire has passed the one-valve, a catheter can be pushed through the open valve, optionally over guide wire 100.

The element 106 width in its collapsed state is about the same as the width 112 of guide-wire 100. However, width 112 may be adapted to the operation or to the vessels' diameter. Width 112 may also be adjustable, for example, by twisting or untwisting duct 102. Width 112 may also be a fixed width of about 0.018 inch.

The element 106 may also comprise a collapsible wire mesh. The mesh may be twisted in the collapsed state and untwisted in the expanded state. For example, by pulling the control wire 108, the mesh is pressed against distal end of duct 102 and expands. The mesh expansion may be by unwinding the spiraled grid wires of contract expandable element 106 as the mesh is pulled by control wire 108. The collapsed mesh may be elongated relative to the mesh in the expanded state.

The wire mesh may be replaced, at least partially, by other extendable/contractible mechanism mechanisms. For example, an inflatable balloon, or other extendable/contractible mechanisms, for example, an elastic elements or elements that may be operated by the control wire, for example, as schematically illustrated in FIG. 4C and in FIG. 4D.

FIG. 4C schematically illustrates a side view of a distal end of a guide-wire 120 (similar to guide-wire 100 of FIGS. 4A and 4B) with expandable and contractible elements 122, connected to elastic elements 124, in a collapsed state, and FIG. 4D schematically illustrates the side view in an expanded state.

Element 122 comprises a plurality of elements around the distal end of walls 118 of duct 102. Elements 122 are rotatable about pivot 128 on walls 118 of duct 102 and connected to elastic elements 124 which normally push inwards into lumen 110 in directions 126.

Elements 122 touch tip 104 firmly due to the pressure force of elastic elements 124. Elements 122 and tip 104 are shaped such that when control wire 108 is pulled towards the proximal end of guide-wire 120, tip 104 moves towards lumen 110, while forcing and pushing elements 122 outwards in directions 130 against the pressure of elastic elements 124. Expanded elements 122 push against a vein's wall, stretching and widening the wall and compelling a nearby one-way valve 13 to open. Pushing control wire 108 towards the distal end, control wire 108 pushes tip 104 while elements 122 contract under the pressure force of elastic elements 124 in directions 126, letting guide-wire 120 pass through the open one-way valve.

Other mechanisms may also be used to expand the walls of a vein, for example, a piezoelectric element that expands by voltage passed by a wire in the catheter, or an element comprising a shape memory alloy (SMA) expanding and/or contracting responsive to temperature, for example, by injections into lumen 110 a liquid, such as saline or plasma, at different temperatures

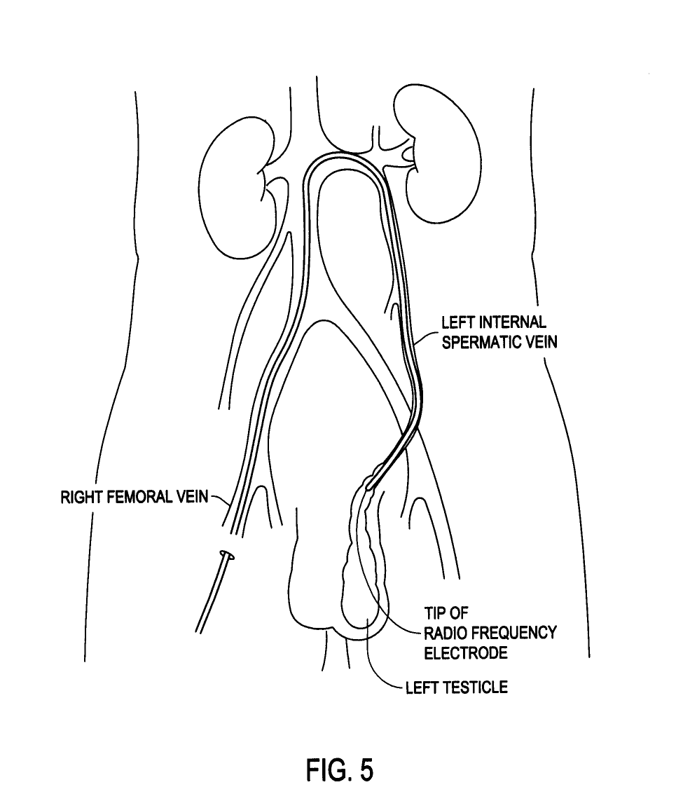

Once the one-way valves have been opened, for example, as described above, the RF probe may be guided through the testicular drainage system to a sufficiently close position to the portion of the vein of the testicular drainage system to be ablated, for example, as depicted in FIG. 5.

Alternatively, direct access to the testicular drainage system using ultrasound guidance to allow direct insertion of the RF probe in the vein of the testicular drainage system to be ablated may be performed.

After at least a portion of one or more of the veins of the testicular drainage system have been ablated, the hydrostatic pressure in the testicular drainage system and/or the testosterone in the testicular drainage system may be measured as discussed above. For example, if the ISV 12 is ablated, the hydrostatic pressure and/or the testosterone concentration may be measured below the ablated portion. Measuring the hydrostatic pressure and/or the testosterone concentration after the ablation may be used to determine if the ablation was effective in reducing the hydrostatic pressure and/or the testosterone concentration in the testicular drainage system and/or the prostatic drainage system. For example, if the hydrostatic pressure and/or the testosterone concentration is not reduced, ablation of additional portions of the testicular drainage system may be performed until the hydrostatic pressure and/or the testosterone concentration in the testicular drainage system, the prostatic drainage system, and/or the prostate is reduced.

It will be obvious to those skilled in the art that various changes and modifications may be made without departing from the spirit and scope of the present disclosure. In addition, illustrated embodiments need not have all the aspects or advantages of the disclosure shown. An aspect or an advantage described in conjunction with a particular embodiment of the present disclosure is not necessarily limited to that embodiment, and can be practiced in any other embodiments described herein, even if not so illustrated. Thus, the present disclosure is intended to cover alternatives, modifications, and equivalents that may fall within the spirit and scope of the present disclosure, as defined by the claims. Accordingly, it is not intended that the disclosure be limited to the specific embodiments described above, except as defined by the claims.

What is claimed is:

1. A method, wherein the method comprises:
identifying a male in need of ablation of at least a portion of one or more veins of a testicular drainage system; and
ablating at least a portion of one or more veins of the testicular drainage system of a human.

2. The method of claim 1, wherein at least a portion of at least one of the veins of the testicular drainage system is an internal spermatic vein.

3. The method of claim 1, wherein the ablating is performed by applying energy to an inside of the one or more veins of the testicular drainage system.

4. The method of claim 3, wherein the energy is radiofrequency energy.

5. The method of claim 1, wherein the identifying comprises identifying a male suffering from benign prostatic hyperplasia, and the ablation is for a treatment of benign prostatic hyperplasia.

6. The method of claim 1, wherein the method further comprises,
before the ablation, measuring an amount of testosterone in at least one of the veins of the testicular drainage system, and measuring an amount of testosterone in a peripheral blood or in prostate tissue.

7. The method of claim 6, wherein the method further comprises after the ablation, measuring the amount of testosterone in at least one of the veins of the testicular drainage system, and measuring the amount of testosterone in the peripheral blood, or in prostate tissue.

8. The method according to claim 6, wherein the measuring includes measuring an amount of free testosterone in the prostatic drainage system.

9. The method of claim 1, wherein the method further comprises, before the ablation, measuring a hydrostatic pressure in at least one of the veins of the testicular drainage system.

10. The method of claim 9, wherein the method further comprises, after the ablation, measuring the hydrostatic pressure in at least one of the veins of the testicular drainage system.

11. A method for the treatment of benign prostatic hyperplasia, the method comprising radiofrequency ablating a least a portion of at least one vein of a testicular drainage system in a male suffering from benign prostatic hyperplasia.

12. The method of claim 11, wherein the portion of the at least one vein of the testicular drainage system is at least a portion of at least one internal spermatic vein.

13. The method of claim 11, wherein the portion of the at least one vein of the testicular drainage system is at least a portion of at least one deferential vein.

14. The method of claim 11, wherein the portion of the at least one vein of the testicular drainage system is at least a portion of at least one internal spermatic vein and at least a portion of at least one deferential vein.

15. A method for the treatment of hyperplasia of a prostate, the method comprising ablating a least a portion of at least one vein of a testicular drainage system in a male suffering from hyperplasia of the prostate, wherein the ablation is performed using a radiofrequency ablation device.

16. The method of claim 15, wherein the method further comprises, before the ablation, measuring a hydrostatic pressure in the least one vein of the testicular drainage system.

17. The method of claim 15, wherein the method further comprises measuring a testosterone level in prostate tissue before the ablation; measuring the testosterone level in prostate tissue after the ablation; and comparing the measured testosterone levels in prostate tissue before and after the ablation.

18. The method of claim 16, wherein the method further comprises, after the ablation,
measuring an amount of testosterone in at least one of the veins of the testicular drainage system;
measuring an amount of testosterone in a peripheral blood; and
comparing the amount of testosterone in the at least one vein of the testicular drainage system and in the peripheral blood.

19. The method of claim 15, wherein the method further comprises, before the ablation,
measuring an amount of testosterone in at least one of the veins of the testicular drainage system;
measuring an amount of testosterone in a peripheral blood; and
comparing the amount of testosterone in the at least one vein of the testicular drainage system and in the peripheral blood.

20. The method according to claim 15, wherein the method further comprises, after the ablation, measuring the hydrostatic pressure in the at least one vein of the testicular drainage system.
